(19) United States
(12) Patent Application Publication
(10) Pub. No.: US 2023/0263550 A1
(43) Pub. Date: Aug. 24, 2023

(54) NEEDLE DRIVE DEVICE

(71) Applicant: LSI Solutions, Inc., Victor, NY (US)

(72) Inventors: Jude S. Sauer, Pittsford, NY (US); Jacob T. Hammond, Rochester, NY (US)

(21) Appl. No.: 18/110,743

(22) Filed: Feb. 16, 2023

Related U.S. Application Data
(60) Provisional application No. 63/311,943, filed on Feb. 18, 2022.

(51) Int. Cl. A61B 17/34

(52) U.S. Cl. CPC A61B 17/3401; A61B 2017/0042

(57) ABSTRACT

A device includes a push rod that extends through a portion of a shaft, and an end of the push rod is translated by rotating a lever. The device further includes a needle assembly coupled to an end of the shaft, and the needle assembly includes a first needle arm and a second needle arm that are pivotable between a first open position and a second closed position. When the lever is rotated by a user, the end of the push rod is configured to contact a portion of the needle assembly to displace the needle assembly from a first open position into a second closed position.

### CROSS-REFERENCE TO RELATED APPLICATIONS

This application claims the benefit of U.S. Provisional Pat. Application No. 63/311,943, filed Feb. 18, 2022, which is incorporated by reference herein in its entirety.

### BACKGROUND OF THE INVENTION

The dura mater is the dense, leathery membrane covering and protecting the spinal cord and spinal nerves, and it is rich in blood vessels. Several surgical procedures involve cutting through the dura mater, such as the repair of a patient’s herniated disc, and closing the dura mater after the procedure is complete. However, there are several risks associated with the opening of the dura mater. Specifically, opening the dura mater can lead to bleeding, which can be difficult to control and can increase the risk of complications. In addition, the dura mater contains and holds the cerebrospinal fluid (CSF) that surrounds the spinal cord, and leakage of the CSF through an opening can cause headaches, nausea, and other symptoms. Accordingly, it is imperative that the opening of the dura mater is securely closed during a procedure to prevent the leakage of blood and CSF. Further, because the dura mater protects the spinal cord and spinal nerves, opening this protective layer can lead to damage to these structures, which could possibly result in permanent nerve damage and loss of function. Accordingly, precise opening, tissue removal and manipulation, and closing (e.g., suturin32g) is critical to avoid damaging spinal structures.

### BRIEF SUMMARY

A device includes a housing portion having a grip portion adapted to be grasped by a hand of a user, and a lever is pivotably coupled to a first portion of the housing portion, the lever being configured to be pivoted between a first lever position and a second lever position. The device also includes a shaft extending along a shaft axis from a proximal end to a distal end, wherein the proximal end of the shaft is coupled to a second portion of the housing portion. The device additionally includes a push rod that extends along a push rod axis from a proximal end to a distal end, wherein at least a portion of the push rod extends through an interior portion of the shaft. The proximal end of the push rod is coupled to a portion of the lever such that when the lever is in the first lever position, the push rod is in a first rod position, and when the lever is in the second lever position, the push rod is in a second rod position. The device further includes a needle assembly coupled to the shaft at or adjacent to the distal end of the shaft. The needle assembly includes a first needle arm and a second needle arm, and the first needle arm and the second needle arm are pivotable or displaceable between a first open position and a second closed position. The distal end of the push rod is configured to contact a portion of the needle assembly when translating from the first rod position to the second rod position, and the distal end of the push rod contacting the portion of the needle assembly displaces at least one of the first needle arm and the second needle arm to transition from the first open position into the second closed position.

### BRIEF DESCRIPTION OF THE DRAWINGS

FIG. 1 is a side view of an embodiment of a device;

FIG. 2A is a side view of the embodiment of the device of FIG. 1 with a first portion of the housing portion omitted for clarity, and with a lever in a first lever position and a needle assembly in a first open position;

FIG. 2B is a detailed side view of the device of FIG. 2A;

FIG. 2C is a side view of the device of FIG. 1 with a second portion of the housing portion omitted for clarity;

FIG. 3 is perspective view of the detailed side view of the device of FIG. 2A with the lever omitted for clarity;

FIG. 4A is a top FIG. 4B is a top view of the embodiment of the locking lever of FIG. 4A;

view of an embodiment of a locking lever of the device of FIG. 1;

FIG. 4B is a top view of the embodiment of the locking lever of FIG. 4A;

FIG. 4C is a bottom view of the embodiment of the locking lever of FIG. 4A;

FIG. 4D is a top view of the embodiment of the locking lever of FIG. 4A;

FIG. 4E is a bottom perspective view of the embodiment of the locking lever of FIG. 4A;

FIG. 5A is a side view of a distal end of an embodiment of a shaft of the device of FIG. 1;

FIG. 5B is the side view of the distal end of the embodiment of the shaft of FIG. 5A with the shaft omitted for clarity;

FIG. 5C is a perspective view of an embodiment of a needle assembly of the device of FIG. 1;

FIG. 5D is a cross-sectional side view of the distal end of the embodiment of the shaft of FIG. 5A with the needle assembly in the second closed position; and

FIG. 6 is a side view of an embodiment of a device with the lever in the second lever position.

### DETAILED DESCRIPTION OF THE INVENTION

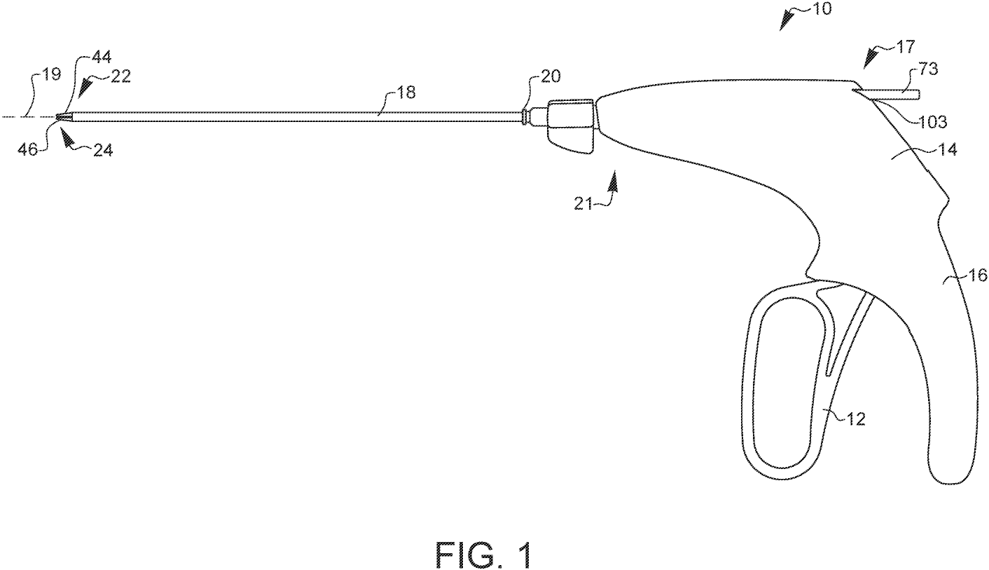

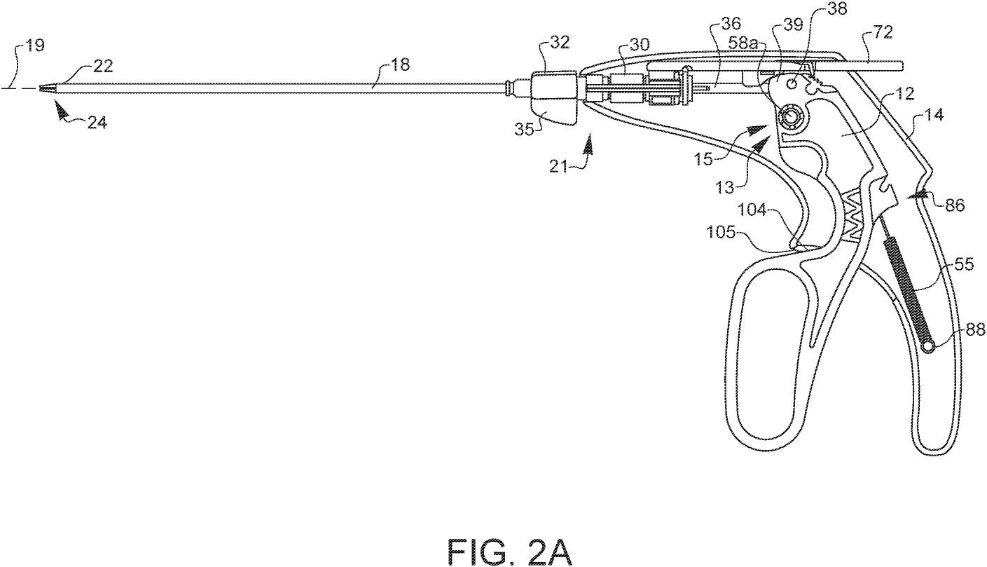

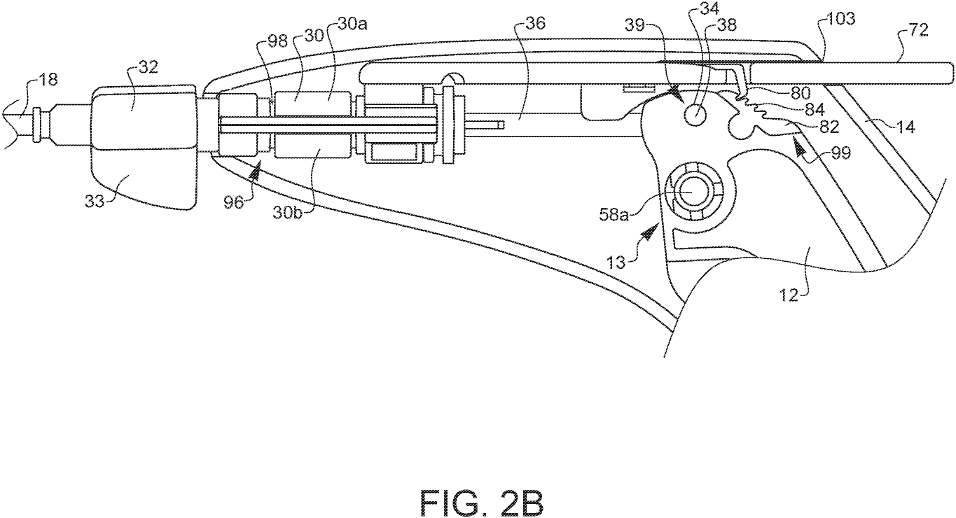

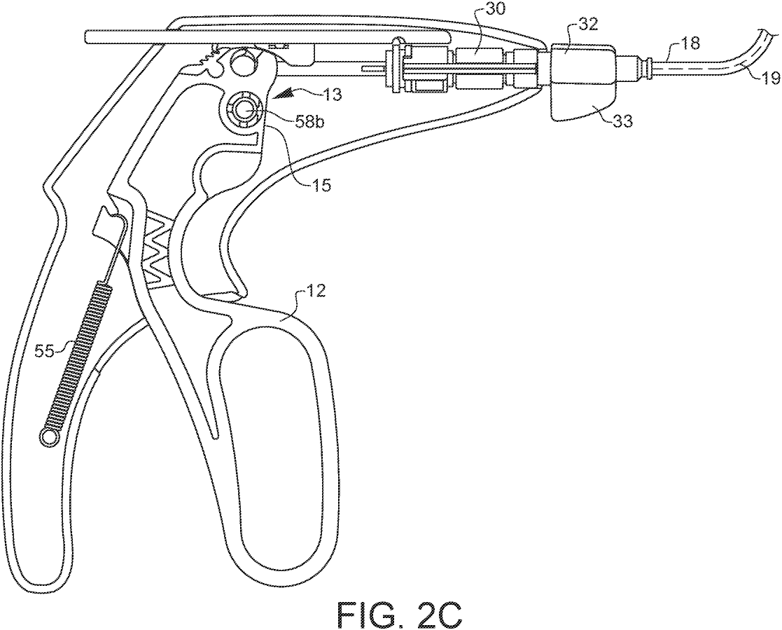

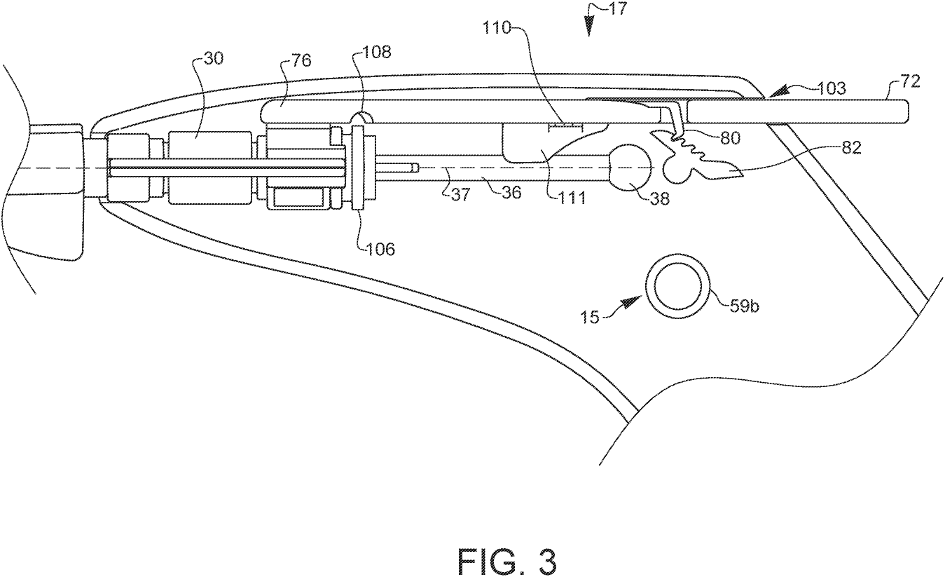

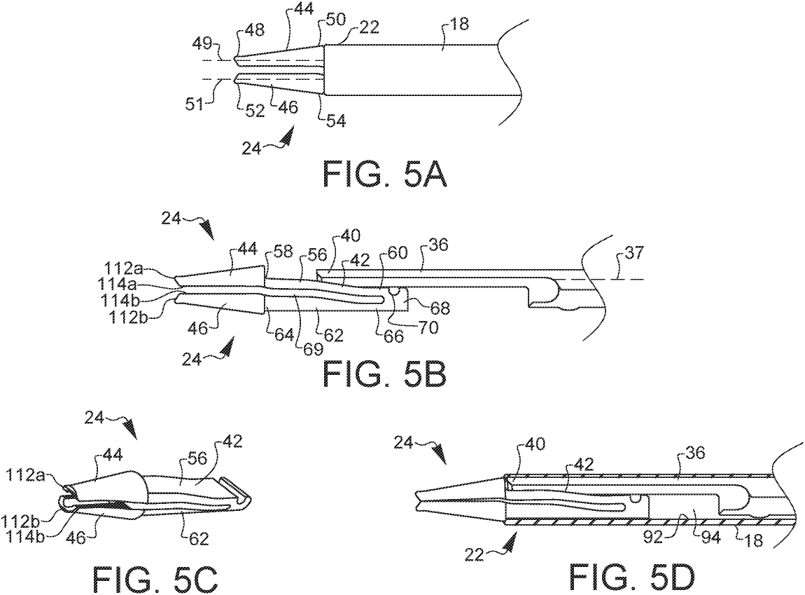

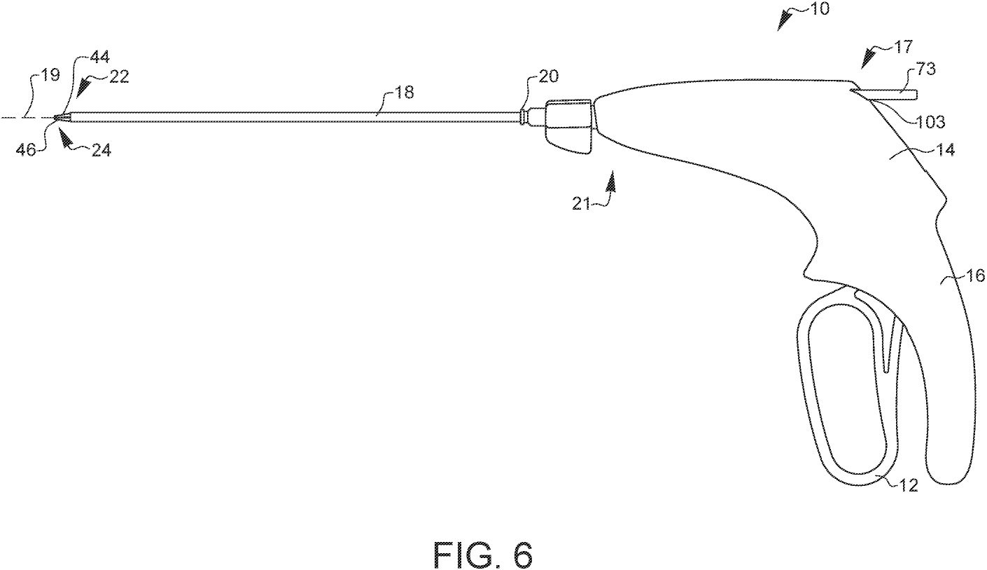

As illustrated in FIG. 1, a needle drive device 10 includes a housing portion 14 having a grip portion 16 adapted to be grasped by a hand of a user. Turning to FIG. 2A, in which a portion of the housing portion 14 is omitted for clarity, a lever 12 is pivotably coupled to a first portion 15 of the housing portion 14, the lever 12 being configured to be pivoted between a first lever position (illustrated in FIG. 1) and a second lever position (illustrated in FIG. 6). The device 10 also includes a shaft 18 extending along a shaft axis 19 from a proximal end 20 to a distal end 22, wherein the proximal end 20 of the shaft 18 is coupled to a second portion 21 of the housing portion 14. As illustrated in FIG. 2B, which is a detailed view of FIG. 2A,, the device 10 additionally includes a push rod 36 that extends along a push rod axis 37 (illustrated in FIG. 3) from a proximal end 38 to a distal end 40 (illustrated in FIG. 5B, in which the shaft 18 is omitted for clarity), wherein at least a portion of the push rod 36 extends through an interior portion 94 (illustrated in FIG. 5D) of the shaft 18. With reference to FIG. 2B, the proximal end 38 of the push rod 36 is coupled to a portion 39 of the lever 12 such that when the lever 12 is in the first lever position, the push rod 36 is in a first rod position (illustrated in FIGS. 5A and 5B), and when the lever 12 is in the second lever position, the push rod 36 is in a second rod position (illustrated in FIG. 5D).

Referring to FIG. 1, the device 10 further includes a needle assembly 24 coupled to the shaft 18 at or adjacent to the distal end 22 of the shaft 18. As illustrated in FIG. 5A, the needle assembly 24 includes a first needle arm 44 and a second needle arm 46, and the first needle arm 44 and the second needle arm 46 are pivotable or displaceable between a first open position (illustrated in FIGS. 5A and 5B) and a second closed position (illustrated in FIG. 5D). Referring to FIG. 5D, the distal end 40 of the push rod 36 is configured to contact a portion (e.g., a portion of a ramp portion 42) of the needle assembly 24 when translating from the first rod position to the second rod position, and the distal end 40 of the push rod 36 contacting the portion of the needle assembly 24 displaces at least one of the first needle arm 44 and the second needle arm 46 to transition from the first open position into the second closed position.

So configured, the device 10 provides an improved ergonomic way to carefully hold small needles using the needle assembly 24 while providing a strong and consistent grasping force that may be locked using a locking mechanism 17. In addition, the compact profile of the first needle arm 44 and the second needle arm 46 of the needle assembly 24 allows for precision placement and grasping of tissue and needles in applications such as suturing.

Turning to the side view of FIG. 1, the device 10 includes the housing portion 14 having a grip portion 16 that is adapted to be grasped by a user to engage and displace a lever 12 from a first lever position (illustrated in FIG. 1) to a second lever position (illustrated in FIG. 6). With reference to FIG. 2A and FIG. 2C (in which a portion of the housing portion 14 is omitted for clarity), a first portion 13 of the lever 12 may be rotatably coupled to the housing portion 14 at a first portion 15 (e.g., a pivot point) of the housing portion 14 such that the lever 12 pivots between the first lever position to the second lever position about the pivot point 15. The lever 12 may pivot about a pivot axis that may extend through the pivot point 15 and may be normal to a shaft axis 19 that extends along the shaft 18 that is coupled to the housing portion 14. In particular, the first portion 13 of the lever 12 may be a pair of aligned bosses 58a, 58b (with 58b illustrated in FIG. 2C) that are received into corresponding cylindrical internal walls (59a not shown, 59b illustrated in FIG. 3, in which a portion of the housing portion 14 and the lever 12 are omitted for clarity) that are each formed on a corresponding interior portion of the housing portion 14 at the pivot point 15 of the housing portion 14. As shown in FIG. 2A, a portion 104 of the lever 12 may contact a portion 105 of the housing portion 14 when the lever 12 is in the first lever position to prevent the lever 12 from overextending beyond the first lever position. A first end of a spring 55 may be coupled to a second portion 86 of the lever 12 and a second end of the spring 55 may be coupled to a portion 88 of the interior portion of the housing portion 14 such that the lever 12 is biased into the first lever position by the spring 55.

Still referring to FIG. 2A, the device 10 includes the shaft 18 that extends from a proximal end 20 to a distal end 22 along the shaft axis 19, and one or more portions of the shaft 18 may be rotatably coupled to a second portion 21 of the housing portion 14 such that the shaft 18 rotates relative to a housing portion 14 about the shaft axis 19. However, in some embodiments, the shaft 18 may be fixed relative to the housing portion 12. As illustrated in FIGS. 1 and 2A, the shaft axis 19 may be linear, or, as illustrated in FIG. 2C, the shaft axis 19 may be non-linear. In other embodiments, one or more portions of the shaft axis 19 may be non-linear. For example, one or more portions of the shaft axis 19 may be linear, or two or more portions of the shaft axis 19 may be non-linear and two or more portions of the shaft axis 19 may be linear. The shaft 18 may be rigid, but in other embodiments, the shaft 18 may be flexible or may have one or more portions that are flexible.

The shaft 18, or one or more portions of the shaft 18, may have the general shape of an elongated hollow tube having an interior surface 92 (illustrated in FIG. 5D) that defines an interior portion 94 (illustrat15ed in FIG. 5D) that extends from the proximal end 20 to the distal end 22 of the shaft 18. The shaft 18 and the interior surface 92 may have any suitable cross-sectional shape or combination of shapes normal to the shaft axis 19. For example, as illustrated in FIG. 2A, the shaft 18 may have the general shape of an elongated cylinder, and the interior surface 92 may have a circular cross-sectional shape when viewed normal to the shaft axis 19.

A rotation knob 32 may be coupled to the shaft 18 such that rotating the rotation knob 32 may rotate the shaft 18 about the shaft axis 19. The rotation knob 32 may be coupled to a portion of the shaft 18 that is adjacent to the proximal end 20 of the shaft 18 and/or may be coupled to the portion of the shaft 18 that is distal to the portion of the housing portion 12 to which the shaft 18 is coupled. All or a portion of the rotation knob 32 may also be coupled to a portion of the housing portion 14, such as the second portion 21 of the housing portion 14. The rotational knob 32 may be configured to simultaneously rotate the shaft 18 and the push rod 36 that is disposed at least partially within the interior portion 94 (illustrated in FIG. 5D) of the shaft 18. The rotation knob 32 can have any suitable shape, and in some embodiments, may be formed as a unitary portion of the shaft 18 itself. The rotation knob 32 may also include a direction indicator 33 that may be fin or protrusion that correlates with a direction of the needle assembly 24 such that the operator of the device 10 can readily orient the housing portion 14 such that the needle assembly 24 faces a desired direction. Because it may not always be possible or ergonomically practical for a surgeon to rotate the housing portion 14 of the device, embodiments having a rotatable shaft 18 may offer more orientation flexibility to the surgeon, thereby enabling tissue and prosthetic protection.

With reference to FIG. 2B, a portion 96 of the rotation knob 32 and/or a portion of the shaft 18 (e.g., a portion of the proximal end 20 of the shaft 18) may extend into the interior of the housing portion 14 and the portion 96 of the rotation knob 32 (and/or the portion of the shaft 18) may include two or more facets 98 (described and illustrated in U.S. Pat. No. 10,939,904, the contents of which is incorporated by reference herein in its entirety). The facets may be planar circumferential surfaces of the portion 96, and the facets 98 may cooperate to form a polygonal cross-sectional shape (e.g., a hexagon, octagon, decagon, or dodecagon) when viewed along the shaft axis 19. In other embodiments, the facets 98 may be recesses, bumps, and/or angled edges. The facets 98 may be configured to engage corresponding engagement surfaces of a constraint assembly 30 that is disposed around the facets 98, and the engagement surfaces may correspond in cross-sectional shape with the facets 98. The constraint assembly 30 may be fixedly coupled to one or more interior portions of the housing portion 14. The constraint assembly 30 may include an upper constraint 30a and a lower constraint 30b that is coupled to the upper constraint 30a to form the constraint assembly 30. The constraint assembly 30 may be made from an injection-molded plastic material that may deform under stress.

When each facet 98 is flat against a corresponding engagement surfaces of the constraint assembly 30, rotation of the shaft 18 (to which the rotation knob 32 is coupled) within the constraint assembly 30 is resisted when a torque is applied to the shaft 18 that is below a threshold strength. However, when a sufficiently strong torque is applied to the shaft 18, the rotation of the facets 98 causes the corners between adjacent facets 98 to apply a force on the engagement surfaces of the constraint assembly 30, thereby deflecting or deforming the engagement surfaces and allowing the facets 98 and the shaft 18, to rotate relative to the engagement surfaces of the constraint assembly 30 until the next adjacent facet is flat against the corresponding engagement surface of the constraint assembly 30, thereby allowing the shaft 18 to rotate in specific increments that correspond to the angular spacing of the facets 98 about the shaft axis 19. The mating of the facets 98 with the engagement surfaces of the constraint assembly 30 may be felt by the user, thereby enabling indexing of the shaft along specific rotation positions.

Still referring to FIG. 2A, the device 10 also includes a push rod 36 extending along a push rod axis 37 (illustrated in FIG. 5B) from a proximal end 38 to a distal end 40 (illustrated in FIG. 5B). The push rod 36 is elongated and at least a portion of the push rod 36 extends through the interior portion 94 (illustrated in FIG. 5D) of the shaft 18. As illustrated in FIG. 2B the proximal end 38 of the push rod 36 is coupled to a third portion 39 of the lever 12 such that when the lever 12 is in the first lever position, the push rod 36 is in a first rod position, as illustrated in FIG. 5B. When the lever 12 is pivoted to the second lever position, (illustrated in FIGS. 5D and 6) the push rod 36 is in a second rod position. The push rod 36 may translate linearly or substantially linearly from the first rod position to the second rod position along (or substantially along) the push rod axis 37, and the push rod axis 37 may extend parallel to (or substantially parallel to) the shaft axis 19.

As illustrated in FIG. 2B, the proximal end 38 of the push rod 36 may be coupled to the third portion 39 of the lever 12 in any suitable manner. For example, the proximal end 38 of the push rod 36 may be coupled to the third portion 39 of the lever 12 in a manner that allows the push rod 36 to move relative to the third portion 39 of the lever 12 and/or to rotate about the push rod axis 37 relative to the third portion 39 of the lever 12. In particular, the third portion 39 of the lever 12 may include a socket 34 and the proximal end 38 of the push rod 36 may include a ball-shaped end 90 that is adapted to be rotatably received within the socket 34 such that the ball-shaped end 90 of the push rod 36 may rotate within the socket 34. The push rod 36 may be a single component or multiple components coupled together to form the push rod 36. The push rod 36 may be made of a flexible material to allow the push rod 36 to bend when the shaft 18 is bent or angularly displaced.

When the lever 12 is in the first lever position shown in FIG. 2A, the push rod 36 is in the first rod position in which the distal end 40 of the push rod 36 is disposed a first distance from the distal end 22 of the shaft 18 (illustrated in FIGS. 5A and 5B). In this position, the distal end 40 of the push rod 36 is remote from, and therefore not engaging, a ramp portion 42 of the needle assembly 24, as illustrated in FIG. 5B. However, if the lever 12 is squeezed towards the grip portion 16 of the housing portion 14, the actuator socket 34 will advance the push rod 36 (and therefore the distal end 40 of the push rod 36) towards the distal end 22 of the shaft 18, thereby enabling the distal end 40 of the push rod 36 to engage the ramp portion 42 of the needle assembly 24, as will be described in more detail below. Accordingly, when the push rod 36 is in the first rod position, the portion of the push rod 36 that extends through the interior portion 94 (illustrated in FIG. 5D) of the shaft 18 may be a portion of the push rod 36 from the distal end 40 the push rod 36 to a point distal to the proximal end 38 of the push rod 36. Further, when the push rod 36 is in the second rod position, the portion of the push rod 36 that extends through the interior portion 94 (illustrated in FIG. 5D) of the shaft 18 may be a portion of push rod 36 between the distal end 40 of the push rod 36 and the proximal end 38 of the push rod 36.

The push rod 36 may be configured to slide through a central aperture of the rotation knob 32 in a direction along the shaft axis 19. In some embodiments, the push rod 36 may have one or more keyed features which may slide longitudinally in a mating fashion within a corresponding one or more slots in the central aperture rotation knob 32. When the rotation knob 32 is rotated, however, the one or more keyed features engaged the corresponding one or more slots to rotationally couple the push rod 36 to the rotation knob 32. Because the rotation knob 32 is also coupled to the shaft 18, both the push rod 36 and the shaft 18 may be rotated directly by the rotation knob 32. In other embodiments, the rotation knob 32 may only be rotationally coupled to the shaft 18.

As illustrated in FIGS. 5A to 5D, the device 10 also includes the needle assembly 24 that is disposed at or adjacent to the distal end 22 of the shaft 18. The needle assembly 24 may include the first needle arm 44 and the second needle arm 46. With reference to FIG. 5A, the first needle arm 44 extends from a distal end 48 to a proximal end 50 along a first needle arm axis 49, and the proximal end 50 is disposed adjacent to or at the distal end 22 of the shaft. The second needle arm 46 extends from a distal end 52 to a proximal end 54 along a second needle arm axis 51, and the proximal end 54 is disposed adjacent to or at the distal end 22 of the shaft. The first needle arm 44 and the second needle arm 46 may cooperate to form a frustoconical shape when in the second closed position, and a cross-sectional diameter of the frustoconical shape may gradually decrease from the proximal ends 50, 54 to the distal ends 48, 52. The largest diameter of the frustoconical shape may be less than an outer diameter of the shaft 18 and/or may be greater than an diameter of the interior surface 92 of the shaft 18. A tip portion 112a, 112b at the distal end 48, 52 of each of the first needle arm 44 and the second needle arm 46 may be planar or substantially planar and may not be normal to the first needle arm axis 49 or the second needle arm axis 51. As illustrated in FIG. 5B, the planar surfaces of the tip portion 112a, 112b may be angled inwards as to form a concave V-shape when viewed normal to the shaft axis 19. An engagement surface 114a, 114b of each of the first needle arm 44 and the second needle arm 46 may be planar or substantially planar such that the engagement surfaces mate 114a, 114b when the first needle arm 44 and the second needle arm 46 are in the second closed position. Further, one or more surface features, such as grooves, channels, or protrusions, may be formed on all or a part of the engagement surfaces mate 114a, 114b, as illustrated on engagement surface 114b in FIG. 5C, to securely grip tissue or an object between the first needle arm 44 and the second needle arm 46 when the first needle arm 44 and the second needle arm 46 are in the second closed position.

Turning to FIG. 5B, the needle assembly 24 includes a first support arm 56 coupled to the first needle arm 44 and a second support arm 62 coupled to the second needle arm 46. The first support arm 56 extends from a distal end 58 to a proximal end 60 along a first support arm axis, and the second support arm 62 extends from a distal end 64 to a proximal end 66 along a second support arm axis. The distal end 58 of the first support arm 56 may be coupled to the proximal end 50 of first needle arm 44 such that the first support arm 56 generally extends along the first needle arm axis 49. In addition, the distal end 64 of the second support arm 62 may be coupled to proximal end 54 of the second needle arm 46 such that the second support arm 62 generally extends along the second needle arm axis 51. The first support arm 56 and the second support arm 62 may be disposed within the interior portion 94 (illustrated in FIG. 5D) of the shaft 18, and each of the distal end 58 of the first support arm 56 and the distal end 64 of the second support arm 62 may be at or adjacent to the distal end 22 of the shaft 18.

Still referring to FIG. 5B, the proximal end 60 of the first support arm 56 may be coupled to a first portion of a hub portion 68 and the proximal end 66 of the second support arm 62 may be coupled to a second portion of the hub portion 68, and the hub portion 68 may extend substantially normal to the shaft axis 19 such that a gap 69 exists between the first support arm 56 and the second support arm 62 when the needle assembly 24 is in the first open position. However, when the distal end 40 of the push rod 36 is moved distally by the squeezing of the lever 12, the distal end 40 of the push rod 36 contacts or engages the ramp portion 42 of the needle assembly 24. The ramp portion 42 may be a surface on a portion of the first support arm 56 that may extend from a first point proximal to the distal end 58 of the first support arm 56 to a second point distal to the proximal end 60 of the first support arm 56. The ramp portion 42 may be a surface of the first support arm 56 that may be planar or planar and contoured such that in cross-section, the planar surface slopes downwardly from the distal end to the proximal end. Thus, when the distal end 40 of the push rod 36 engages the ramp portion 42 of the needle assembly 24, an inner-facing surface of the first needle arm 44 is displaced towards an inner-facing surface of the second needle arm 46 such that the inner-facing surface of the first needle arm 44 contacts (or is directly adjacent to) the inner-facing surface of the second needle arm 46 (and moves into the closed position of FIG. 5D) such that tissue or an object, such as a needle, may be captured between the first needle arm 44 and the second needle arm 46. The needle assembly 24 may be secured to the shaft 18 in any suitable manner, and a pin (not shown) may extend through an aperture 70 formed in the hub portion 68, and the pin may be secured to one or more portions of the shaft 18 disposed at or adjacent to the distal end 22.

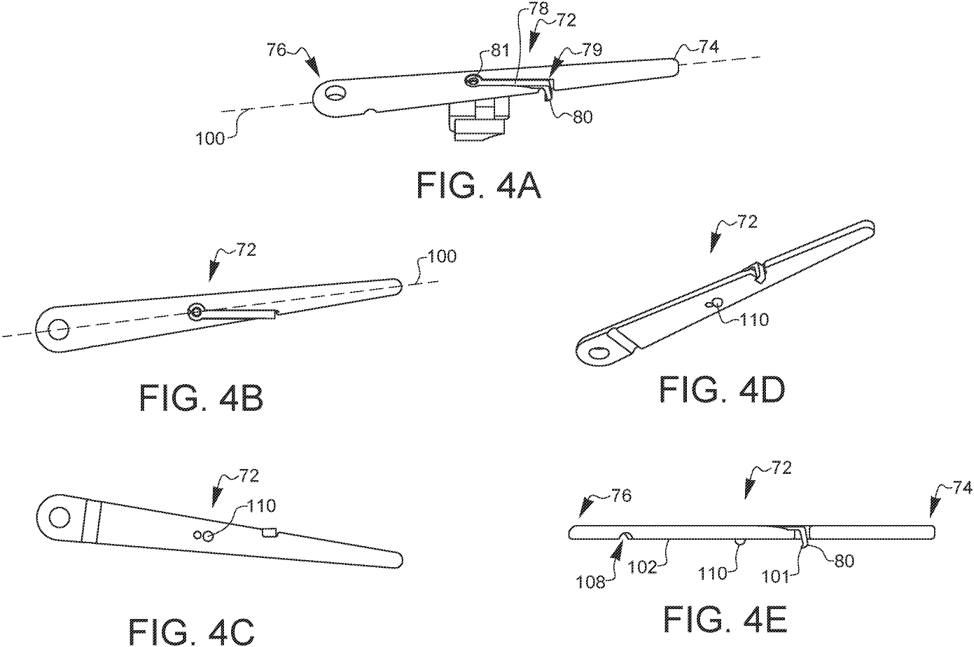

Turning to FIGS. 1 and 2A, the device 10 may include a locking mechanism 17 configured to lock the needle assembly 24 in one or more positions, such as in the second closed position (illustrated in FIG. 5D) and/or one or more intermediate positions between the closed position and the first open position of FIGS. 5A and 5B. In particular, with reference to FIG. 2B, the locking mechanism 17 may include an elongated lock lever 72 having a pawl arm 80 that is configured to be moved into an out of engagement with one or more teeth 84 of a ratchet member 82 that may be coupled to a portion of the lever 12 (or to a portion of the housing portion 14). For example, the ratchet member 82 may be coupled to a fourth portion 99 of the lever 12 that is adjacent to the third portion 39 of the lever 12. As illustrated in FIGS. 4A to 4E, which illustrate various views of the lock lever 72, the lock lever 72 may extend along a lock member axis 100 from a first end 76 to a second end 74, and the lock lever 72 may be planar or substantially planar. An elongated locking element 78 may be coupled to the lock lever 72, and the locking element 78 may extend from a first end 81 to a second end 79 along an axis that is not parallel to the lock member axis 100, and the pawl arm 80 may downwardly extend from the second end 79 of the locking element 78. The pawl arm 80 may extend in a notch formed in a lateral surface of the lock lever 72, and an end portion 101 of the pawl arm 80 may extend beyond a bottom surface 102 of the lock lever 72 (illustrated in FIG. 4E). As illustrated in FIG. 3, the lock lever 72 (at or adjacent to the first end 76) may be rotatably coupled to a portion of the housing portion 14 or a portion of the constraint assembly 30, such as by a journal, and a lever pivot axis may be normal to the shaft axis 19 and/or the pivot axis of the lever 12. A user may pivot the lock lever 72 about the lever pivot axis by grasping a portion of the second end 74 that extends proximally through a slot 103 formed in a proximal portion of the housing portion 14 and pivoting the lock lever 72 from a disengaged position (not shown) to an engaged position (FIG. 2B) and vice versa. Because there may be more than one tooth 84 on the ratchet member, each tooth 84 may correspond to a specific separation distance between the first needle arm 44 and the second needle arm 46 to allow for multiple locking positions of the locking mechanism 17.

When the lever 12 is in a position corresponding to a desired position of the needle assembly 24 (such as the second lever position in which the needle assembly 24 is in the second closed position), the lock lever 72 may be rotated from the disengaged position to the engaged position to maintain the lever 12 in the position (i.e., a desired position) corresponding to the desired position of the needle assembly 24. With the lever 12 in the desired position, and with the lock lever 72 pivoted from the disengaged position to the engaged position, the end portion 101 of the pawl arm 80 engages or is disposed closely adjacent to a corresponding one of the plurality of teeth 84 of the ratchet member 84. With the end portion 101 of the pawl arm 80 so positioned, the spring 55 (illustrated in FIG. 2A) biases the lever 12 in a rotational direction such that the plurality of teeth 84 of the ratchet member 82 tend to move or rotate proximally relative to the pawl arm 80, and the one or more of the teeth 84 of the ratchet member 82 rotates into direct engagement with the end portion 101 of the pawl arm 80 to prevent the lever 12 from rotating from the desired position into the first lever position and “locking” the lever 12 in the desired position. If the locking lever 72 is rotated into the engaged position at a lever position in which the needle assembly 24 is between the first open position and the second closed position, the needle assembly 24 will be locked in that intermediate position.

To release the “lock” and displace the lock lever 72 from the engaged position to the disengaged position, the user slightly squeezes the lever 12 to release or relieve the tension on the pawl arm 80 caused by the engagement of the one or more of the teeth 84 of the ratchet member 82. With the tension relived, the lock lever 72 can be pivoted from the engaged position to the disengaged position, thereby allowing the lever 12 to return to the first lever position of FIG. 1 in which the distal end 40 of the push rod 36 is not engaging a ramp portion 42 of the needle assembly 24, thereby allowing the needle assembly 24 to move to the open position of FIG. 5A.

In addition, as illustrated in FIG. 3, when pivoting the lock lever 72 from the disengaged position to the engaged position, a top portion of an annular member 106 disposed at or slightly offset from a proximal end of the constraint assembly 30 may be received into a guide groove 108 formed in the bottom surface of the lock lever 72 to ensure correct positioning of the lock lever 72 relative to the ratchet member 88. A detent feature 110, such as a hemispherical protrusion, disposed on the bottom surface 102 of the lock lever 72 may also be received into a corresponding receiving feature 111 to positively “snap” the lock lever 72 into the first lever position.

In use, and with reference to FIG. 1, a user (such as a surgeon or any other medical professional) may grasp the grip portion 16 of the housing portion 14 and position the first needle arm 44 and the second needle arm 46 of the needle assembly 24 in a desired location, such as around an object that is to be grasped (for example, around tissue or a needle). The user then displaces the lever 12 from the first lever position to the second lever position (illustrated in FIG. 6). As described herein, displacing the lever 12 to the second lever position causes the distal end 40 of the push rod 36 to engage the ramp portion 42 of the needle assembly 24, as illustrated in FIG. 5D, which in turn causes the engagement surfaces 114a, 114b (see FIG. 5B) of the first needle arm 44 and the second needle arm 46 to engage the object. Once engaged, the user may pivot the lock lever 72 into an engaged position in which the object is secured between the first needle arm 44 and the second needle arm 46. When the user desires to release the object, the lock lever 72 may be pivoted from the engaged position into the disengaged position, thereby allowing the first needle arm 44 and the second needle arm 46 to move to the open position of FIG. 5A. In some embodiments the lock lever 72 is configured to be pivoted using the same hand of the user that grasps the device 10.

Various advantages of a surgical equipment holder have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A device comprising:
a housing portion having a grip portion adapted to be grasped by a hand of a user;
a lever pivotably coupled to a first portion of the housing portion, the lever configured to be pivoted between a first lever position and a second lever position;
a shaft extending along a shaft axis from a proximal end to a distal end, wherein the proximal end of the shaft is coupled to a second portion of the housing portion;
a push rod extending along a push rod axis from a proximal end to a distal end, wherein at least a portion of the push rod extends through an interior portion of the shaft, wherein the proximal end of the push rod is coupled to a portion of the lever such that when the lever is in the first lever position, the push rod is in a first rod position, and when the lever is in the second lever position, the push rod is in a second rod position; and
a needle assembly coupled to the shaft at or adjacent to the distal end of the shaft, the needle assembly comprising a first needle arm and a second needle arm, wherein the first needle arm and the second needle arm are displaceable between a first open position and a second closed position, wherein the distal end of the push rod contacts a portion of the needle assembly when translating from the first rod position to the second rod position, and wherein the distal end of the push rod contacting the portion of the needle assembly displaces at least one of the first needle arm and the second needle arm to transition the needle assembly from the first open position into the second closed position.

2. The device of claim 1, wherein the push rod translates along the push rod axis from the first rod position to the second rod position when the lever is pivoted from the first lever position to the second lever position.

3. The device of claim 2, wherein the proximal end of the push rod includes a ball-shaped end and the portion of the handle includes a socket that is configured to rotatably receive the ball-shaped end of the push rod such that the push rod is configured to rotate about the push rod axis relative to the lever.

4. The device of claim 1, wherein at least a portion of the shaft is flexible such that at least a portion of the shaft axis is non-linear.

5. The device of claim 1, wherein the shaft is rotatably coupled to the second portion of the housing portion such that the shaft is configured to rotate about the shaft axis relative to the second portion of the housing portion.

6. The device of claim 1, wherein the first needle arm extends from a distal end to a proximal end along a first needle arm axis, and the proximal end of the first needle arm is disposed adjacent to or at the distal end of the shaft, and wherein the second needle arm extends from a distal end to a proximal end along a second needle arm axis, and the proximal end of the second needle arm is disposed adjacent to or at the distal end of the shaft.

7. The device of claim 6, wherein the needle assembly includes a first support arm having a proximal end and a distal end that extends along a first support arm axis, wherein the distal end of the first support arm is coupled to the proximal end of first needle arm such that the first support arm axis generally extends along the first needle arm axis, and
wherein the needle assembly includes a second support arm having a proximal end and a distal end that extends along a second support arm axis, wherein the distal end of the second support arm is coupled to the proximal end of second needle arm such that the second support arm axis generally extends along the second needle arm axis, and
wherein the first support arm and the second support arm are disposed within the interior portion of the shaft.

8. The device of claim 7, wherein the distal end of the push rod contacts a ramp portion of the first support arm to displace at least the first needle arm to transition the needle assembly from the first open position into the second closed position.

9. The device of claim 7, wherein the proximal end of the first support arm is coupled to a first portion of a hub portion and the proximal end of the second support arm is coupled to a second portion of the hub portion, and the hub portion extend substantially normal to the shaft axis such that a gap exists between the first support arm and the second support arm when the needle assembly is in the first open position.

10. The device of claim 1, the lever further comprising a ratchet member having a plurality of teeth and the device further comprising a locking mechanism, the locking mechanism comprising;
an elongated lock lever pivotably coupled to a portion of the housing portion;
a pawl arm coupled to a portion of the lock lever, the pawl arm having an end portion that extends beyond a bottom surface of the lock lever, wherein the lock lever is configured to be pivoted between an engaged position to a disengaged position,
wherein in the engaged position, the end portion of the pawl arm engages one of the plurality of teeth of the ratchet member to maintain the lever is a desired position, and
in the disengaged position, the end portion of the pawl arm does not engage one of the plurality of teeth of the ratchet member.

11. A device comprising:
a housing portion having a grip portion adapted to be grasped by a hand of a user;
a lever pivotably coupled to a first portion of the housing portion, the lever configured to be pivoted between a first lever position and a second lever position;
a shaft extending along a shaft axis from a proximal end to a distal end, wherein the proximal end of the shaft is rotatably coupled to a second portion of the housing portion such that the shaft is configured to rotate about the shaft axis relative to the second portion of the housing portion;
a push rod extending along a push rod axis from a proximal end to a distal end, wherein at least a portion of the push rod extends through an interior portion of the shaft, wherein the proximal end of the push rod is coupled to a portion of the lever such that when the lever is in the first lever position, the push rod is in a first rod position, and when the lever is in the second lever position, the push rod is in a second rod position, wherein the push rod translates along the push rod axis from the first rod position to the second rod position when the lever is pivoted from the first lever position to the second lever position, and wherein the proximal end of the push rod includes a ball-shaped end and the portion of the handle includes a socket that is configured to rotatably receive the ball-shaped end of the push rod such that the push rod is configured to rotate about the push rod axis relative to the lever; and
a needle assembly coupled to the shaft at or adjacent to the distal end of the shaft, the needle assembly comprising a first needle arm and a second needle arm, wherein the first needle arm and the second needle arm are displaceable between a first open position and a second closed position, wherein the distal end of the push rod contacts a ramp portion of the needle assembly when translating from the first rod position to the second rod position, and wherein the distal end of the push rod contacting the portion of the needle assembly displaces at least one of the first needle arm and the second needle arm to transition the needle assembly from the first open position into the second closed position,
wherein the first needle arm extends from a distal end to a proximal end along a first needle arm axis, and the proximal end of the first needle arm is disposed adjacent to or at the distal end of the shaft, and wherein the second needle arm extends from a distal end to a proximal end along a second needle arm axis, and the proximal end of the second needle arm is disposed adjacent to or at the distal end of the shaft,
wherein the needle assembly includes a first support arm having a proximal end and a distal end that extends along a first support arm axis, wherein the distal end of the first support arm is coupled to the proximal end of first needle arm such that the first support arm axis generally extends along the first needle arm axis,
wherein the needle assembly includes a second support arm having a proximal end and a distal end that extends along a second support arm axis, wherein the distal end of the second support arm is coupled to the proximal end of second needle arm such that the second support arm axis generally extends along the second needle arm axis, and
wherein the first support arm and the second support arm are disposed within the interior portion of the shaft, and
wherein the proximal end of the first support arm is coupled to a first portion of a hub portion and the proximal end of the second support arm is coupled to a second portion of the hub portion, and the hub portion extend substantially normal to the shaft axis such that a gap exists between the first support arm and the second support arm when the needle assembly is in the first open position.

12. The device of claim 11, the lever further comprising a ratchet member having a plurality of teeth and the device further comprising a locking mechanism, the locking mechanism comprising;
an elongated lock lever pivotably coupled to a portion of the housing portion;
a pawl arm coupled to a portion of the lock lever, the pawl arm having an end portion that extends beyond a bottom surface of the lock lever, wherein the lock lever is configured to be pivoted between an engaged position to a disengaged position,
wherein in the engaged position, the end portion of the pawl arm engages one of the plurality of teeth of the ratchet member to maintain the lever is a desired position, and
in the disengaged position, the end portion of the pawl arm does not engage one of the plurality of teeth of the ratchet member.

13. The device of claim 12, wherein the shaft axis is linear.

14. The device of claim 12, wherein at least a portion of the shaft is flexible such that at least a portion of the shaft axis is non-linear.
